(19) United States
(12) Patent Application Publication
(10) Pub. No.: US 2022/0056510 A1
(43) Pub. Date: Feb. 24, 2022

(54) ENZYMATIC TABLET AND USES THEREOF

(71) Applicant: Detect, Inc., Guilford, CT (US)

(72) Inventor: Jonathan M. Rothberg, Miami Beach, FL (US)

(73) Assignee: Detect, Inc., Guilford, CT (US)

(21) Appl. No.: 17/406,730

(22) Filed: Aug. 19, 2021

Related U.S. Application Data
(60) Provisional application No. 63/161,607, filed on Mar. 16, 2021.
(60) Provisional application No. 63/068,303, filed on Aug. 20, 2020.

(51) Int. Cl. C12Q 1/6848

(52) U.S. Cl. CPC C12Q 1/6848

(57) ABSTRACT

The disclosure relates, in some aspects, to compositions and methods for amplifying nucleic acids. In some embodiments, the disclosure describes solid compositions comprising a first enzyme (e.g., a reverse transcriptase) and a second enzyme (e.g., a polymerase), and optionally a third enzyme (e.g., a Uracil-DNA glycosylase), where each enzyme is under the control of a molecular switch. In some embodiments, solid compositions described by the disclosure allow for single-tube, temperature-controlled lysis, decontamination, and amplification of nucleic acid s (e.g., DNA or RNA) from a biological sample without the need to add additional reaction components or transfer the reaction mixture from one container to another.

### RELATED APPLICATIONS

This Application claims the benefit under 35 U.S.C. 119(e) of the filing date of U.S. provisional Application Ser. No. 63/161,607, filed Mar. 16, 2021, entitled “ENZYMATIC TABLET AND USES THEREOF”, and U.S. provisional Application Ser. No. 63/068,303, filed Aug. 20, 2020, entitled “APPARATUSES AND METHODS FOR PERFORMING RAPID MULTIPLEXED DIAGNOSTIC TESTS”, the entire contents of each of which are incorporated herein by reference.

### BACKGROUND

Nucleic acid-based diagnostic tests often require multiple steps, for example isolation of nucleic acids, reverse transcription, amplification, and detection. Between each of these steps, there is often a need to change buffers or other reagents or transfer the reaction mixture between different containers, which increases the chances that external contaminants could be introduced into the sample.

### SUMMARY

Aspects of the disclosure relate to compositions and methods for amplifying nucleic acids. The disclosure is based, in part, on solid compositions comprising a first enzyme (e.g., a reverse transcriptase) and a second enzyme (e.g., a polymerase), and optionally a third enzyme (e.g., a Uracil-DNA glycosylase), where each enzyme is under the control of a molecular switch. Without wishing to be bound by any particular theory, solid compositions described herein allow for single-tube, temperature-controlled lysis, decontamination, and amplification of nucleic acid s (e.g., DNA or RNA) from a biological sample without the need to add additional reaction components or transfer the reaction mixture from one container to another.

Accordingly, in some aspects, the disclosure provides a solid composition comprising a first enzyme; and a second enzyme, wherein the first enzyme and second enzyme are each under the control of a molecular switch.

In some embodiments, a composition is in the form of a pellet, capsule, gelcap, or tablet.

In some embodiments, a first enzyme is a reverse transcriptase enzyme. In some embodiments, a reverse transcriptase enzyme is a heat-stable reverse transcriptase enzyme. In some embodiments, a reverse transcriptase enzyme is selected from the group consisting of HIV-1 reverse transcriptase, Moloney murine leukemia virus (M-MLV) reverse transcriptase, and avian myeloblastosis virus (AMV) reverse transcriptase, Warmstart RTx, Superscript IV, Maxima RT, Ultrascript 2.0, RapiDxFire, eAMV RT, APExBIO reverse transcriptase, and Transcriptme (R32).

In some embodiments, a second enzyme is a polymerase enzyme. In some embodiments, a polymerase enzyme is a heat-stable polymerase enzyme. In some embodiments, a polymerase is selected from the group consisting of Bacillus stearothermophilus (Bst), Bacillus Smithii (Bsm), Geobacillus sp. M (GspM), Thermodesulfatator indicus (Tin), and Taq DNA polymerase.

In some embodiments, a first enzyme and second enzyme are under the control of the same molecular switch. In some embodiments, a molecular switch comprises an aptamer binding site, an antibody binding site, or a photocleavable site.

In some embodiments, a solid composition further comprises an inactivating agent bound to the molecular switch. In some embodiments, an inactivating agent comprises an aptamer or an antibody. In some embodiments, an aptamer is a polynucleotide aptamer or a peptide aptamer.

In some embodiments, a first enzyme and second enzyme are each under the control of a different molecular switch. In some embodiments, the molecular switch of a first enzyme comprises an aptamer binding site, an antibody binding site, or a photocleavable site. In some embodiments, the molecular switch of a second enzyme comprises an aptamer binding site, an antibody binding site, or a photocleavable site.

In some embodiments, a solid composition further comprises an inactivating agent bound to each molecular switch. In some embodiments, each inactivating agent is independently selected from the group consisting of an aptamer and an antibody. In some embodiments, each aptamer is a polynucleotide aptamer or a peptide aptamer.

In some embodiments, a solid composition further comprises a third enzyme. In some embodiments, a third enzyme is an Uracil-DNA glycosylase (UDG) enzyme. In some embodiments, a UDG enzyme is heat-sensitive. In some embodiments, a UDG enzyme is a bacterial UDG or a mammalian UDG. In some embodiments, a UDG enzyme is a recombinant UDG expressed by E. coli.

In some embodiments, a solid composition further comprises one or more pharmaceutically acceptable excipients.

In some aspects, the disclosure provides a composition comprising a solid composition as described herein and a biological sample comprising DNA or RNA.

In some embodiments, DNA or RNA is derived from one or more pathogens. In some embodiments, one or more pathogens are viral, bacterial, fungal, parasitic, or protozoan pathogens.

In some embodiments, a biological sample comprises blood, saliva, mucus, urine, feces, cerebrospinal fluid (CSF), or tissue.

In some embodiments, a composition further comprises one or more buffering agents. In some embodiments, a composition further comprises a lysis buffer.

In some embodiments, the solid composition dissolves upon contact with a biological sample and/or lysis buffer.

In some embodiments, a composition has a temperature of between about 30° C. and about 40° C. In some embodiments, a composition has a temperature of between about 50° C. and about 70° C.

In some aspects, the disclosure provides a single-tube method for amplification of a target nucleic acid, the method comprising contacting a solid composition as described herein with a biological sample comprising nucleic acids to produce a reaction mixture; incubating the reaction mixture under conditions under which uracil-containing nucleotides are removed from the nucleic acids in the reaction mixture by a UDG enzyme; incubating the reaction mixture under conditions under which the UDG enzyme is inactivated and the molecular switches of the first and/or second enzymes of the solid composition are activated; amplifying a target nucleic acid from the reaction mixture using the first and/or second enzymes.

In some embodiments, step (i) further comprises contacting the solid composition with one or more of the following to produce the reaction mixture: one or more buffering agents, one or more oligonucleotide primers, and/or a population of deoxyribonucleotide triphosphates (dNTPs).

In some embodiments, a biological sample comprises blood, saliva, mucus, urine, feces, cerebrospinal fluid (CSF), or tissue. In some embodiments, a biological sample comprises DNA or RNA. In some embodiments, DNA or RNA is derived from one or more pathogens. In some embodiments, one or more pathogens are viral, bacterial, fungal, parasitic, or protozoan pathogens.

In some embodiments, the conditions of step (ii) comprise incubating the reaction mixture at a temperature ranging from about 30° C. to about 40° C.

In some embodiments, the conditions of step (iii) comprise incubating the reaction mixture at a temperature ranging from about 50° C. to about 70° C.

In some embodiments, a UDG enzyme is a component of the solid composition. In some embodiments, a UDG enzyme is heat-sensitive. In some embodiments, a UDG enzyme is a bacterial UDG or a mammalian UDG. In some embodiments, a UDG enzyme is an E. coli UDG.

In some aspects, the disclosure provides a kit comprising: (i) a container housing a solid composition as described herein; (ii) a container housing one or more buffering agents; (iii) a container housing one or more oligonucleotide primers; and (iv) a container housing a population of deoxyribonucleotide triphosphates (dNTPs).

In some embodiments, the container of (i), (ii), (iii), and (iv) are the same container.

In some embodiments, the container of (i), (ii), (iii), and (iv) are each a different container.

In some embodiments, the first enzyme of the solid composition is a reverse transcriptase enzyme. In some embodiments, a reverse transcriptase enzyme is a heat-stable reverse transcriptase enzyme.

In some embodiments, the second enzyme of the solid composition is a polymerase enzyme. In some embodiments, a polymerase enzyme is a heat-stable polymerase enzyme.

### DETAILED DESCRIPTION

As described herein, a sample (e.g., nucleic acids of a biological sample) may undergo lysis and amplification prior to detection. The reagents associated with lysis, amplification and/or detection may be in solid form (e.g., lyophilized, dried, crystallized, air-jetted, etc.). In certain embodiments, one or more (and, in some cases, all) of the reagents necessary for lysis and/or amplification may be present in a single pellet, capsule, gelcap, or tablet. In some embodiments, the pellet, capsule, gelcap, or tablet may comprise two or more enzymes, and it may be necessary for the enzymes to be activated in a particular order. Therefore, in some embodiments of the present technology, the enzyme-containing tablet, pellet, capsule, or gelcap may further comprise one or more molecular switches.

### Reverse Transcriptases

Aspects of the disclosure relate to diagnostic methods comprising a step of amplifying genetic material from a target pathogen. In some cases, a target pathogen has RNA as its genetic material. In certain instances, for example, a target pathogen is an RNA virus (e.g., a coronavirus, an influenza virus). In some such cases, the target pathogen's RNA may need to be reverse transcribed to DNA prior to amplification.

In some embodiments, reverse transcription is performed by exposing lysate (or nucleic acids within a lysate) obtained from a biological sample to one or more reverse transcription (RT) reagents. In certain instances, the one or more reverse transcription reagents comprise a reverse transcriptase, a DNA-dependent polymerase, and/or a ribonuclease (RNase).

A “reverse transcriptase” generally refers to an enzyme that transcribes RNA to complementary DNA (cDNA) by polymerizing deoxyribonucleotide triphosphates (dNTPs). Examples of RT enzymes include but are not limited to HIV-1 reverse transcriptase, Moloney murine leukemia virus (M-MLV) reverse transcriptase, and avian myeloblastosis virus (AMV) reverse transcriptase. An RT enzyme may be naturally occurring (e.g., isolated from an organism) or synthetic. In some embodiments, synthetic RT enzymes comprise an RT enzyme that has been isolated from an organism and subsequently engineered (e.g., through recombinant DNA methods, mutagenesis, etc.) to comprise one or more amino acid mutations relative to the wild type RT enzyme from which it is derived. Additional examples of RT enzymes include but are not limited to Warmstart RTx, Superscript IV, Maxima RT, Ultrascript 2.0, RapiDxFire, eAMV RT, APExBIO reverse transcriptase, and Transcriptme (R32).

In some embodiments, a RT enzyme is heat-stable. A “heat stable” RT enzyme generally refers to a RT enzyme that retains its function (e.g., reverse transcription of RNA) at temperatures ranging from about 37° C. to 60° C., 37° C. to 70° C., 37° C. to 80° C., 37° C. to 90° C., 40° C. to 60° C., 40° C. to 70° C., 40° C. to 80° C., 40° C. to 90° C., 50° C. to 60° C., 50° C. to 70° C., 50° C. to 80° C., 50° C. to 90° C., 60° C. to 70° C., 60° C. to 80° C., 60° C. to 90° C., 70° C. to 80° C., 70° C. to 90° C., or 80° C. to 90° C.

The concentration or amount of a RT enzyme in a solid composition may vary. In some embodiments, the amount of RT enzyme in a solid composition at least 0.1 U/μL, at least 0.2 U/μL, at least 0.3 U/μL, at least 0.4 U/μL, at least 0.5 U/μL, at least 0.6 U/μL, at least 0.7 U/μL, at least 0.8 U/μL, at least 0.9 U/μL, or at least 1.0 U/μ.L. In some embodiments, the concentration of the RT enzyme is in a range from 0.1 U/μL to 0.5 U/μL, 0.1 U/μL to 1.0 U/μL, 0.2 U/μL to 0.5 U/μL, 0.2 U/μL to 1.0 U/μL, or 0.5 U/μL to 1.0 U/μL. In some embodiments, a “Unit” of RT enzyme is defined as the amount of enzyme required to incorporate 1 nmol of dTTP into acid insoluble material in 10 minutes at 37° C. using poly r(A)/oligo (dT) as a substrate.

In some embodiments, a reverse transcriptase enzyme further comprises a molecular switch. In some embodiments, the molecular switch comprises one or more (e.g., 1, 2, 3, 4, 5, or more) binding site(s) for an agent that regulates activity of the RT enzyme, for example an aptamer binding site, antibody binding site, small molecule binding site, etc. In some embodiments, the molecular switch controlling a reverse transcriptase enzyme is reactive to environmental conditions, for example temperature, pH, salt concentration, etc. For example, in some embodiments, a solid composition comprises a heat stable RT enzyme comprising a molecular switch that is temperature reactive (e.g., an aptamer that binds to, and deactivates, the RT enzyme at temperatures below 65° C., and/or unbinds, and activates, the RT enzyme at temperatures above 65° C.).

### Polymerases

In some embodiments, an amplification buffer comprises a DNA polymerase with high strand displacement activity. Non-limiting examples of suitable DNA polymerases include a DNA polymerase long fragment (LF) of a thermophilic-bacteria, such as Bacillus stearothermophilus (Bst), Bacillus Smithii (Bsm), Geobacillus sp. M (GspM), or Thermodesulfatator indicus (Tin), or a Taq DNA polymerase. In certain embodiments, the DNA polymerase is Bst LF DNA polymerase, GspM LF DNA polymerase, GspSSD LF DNA polymerase, Tin exo-LF DNA polymerase, or SD DNA polymerase. In each case, the DNA polymerase may be a wild type or mutant polymerase.

In some embodiments, a polymerase enzyme is heat-stable. A “heat stable” polymerase enzyme generally refers to a polymerase enzyme that retains its function (e.g., synthesis of DNA strands) at temperatures ranging from about 37° C. to 60° C., 37° C. to 70° C., 37° C. to 80° C., 37° C. to 90° C., 40° C. to 60° C., 40° C. to 70° C., 40° C. to 80° C., 40° C. to 90° C., 50° C. to 60° C., 50° C. to 70° C., 50° C. to 80° C., 50° C. to 90° C., 60° C. to 70° C., 60° C. to 80° C., 60° C. to 90° C., 70° C. to 80° C., 70° C. to 90° C., or 80° C. to 90° C.

In some embodiments, a polymerase enzyme is low temperature inactive. A “low temperature inactive” polymerase enzyme generally refers to a polymerase enzyme that is not active (e.g., does not synthesize of DNA strands) at temperatures ranging below 65° C., 60° C., 55° C., 50° C., 45° C., 40° C., 37° C., or 30° C. In some embodiments, a low temperature inactive polymerase is a Bst polymerase.

In some embodiments, the concentration of the polymerase enzyme is at least 0.1 U/μL, at least 0.2 U/μL, at least 0.3 U/μL, at least 0.4 U/μL, at least 0.5 U/μL, at least 0.6 U/μL, at least 0.7 U/μL, at least 0.8 U/μL, at least 0.9 U/μL, or at least 1.0 U/μL. In some embodiments, the concentration of the polymerase enzyme is in a range from 0.1 U/μL to 0.5 U/μL, 0.1 U/μL to 1.0 U/μL, 0.2 U/μL to 0.5 U/μL, 0.2 U/μL to 1.0 U/μL, or 0.5 U/μL to 1.0 U/μL. In some embodiments, a “Unit” of polymerase enzyme is defined as the amount of enzyme required to incorporate 10 nmol of total deoxyribonucleoside triphosphates into acid precipitable DNA within 60 min at +65 ° C.

In some embodiments, a polymerase enzyme further comprises a molecular switch. In some embodiments, the molecular switch comprises one or more (e.g., 1, 2, 3, 4, 5, or more) binding site(s) for an agent that regulates activity of the polymerase enzyme, for example an aptamer binding site, antibody binding site, small molecule binding site, etc. In some embodiments, the molecular switch controlling a polymerase enzyme is reactive to environmental conditions, for example temperature, pH, salt concentration, etc. For example, in some embodiments, a solid composition comprises a low temperature inactive polymerase enzyme comprising a molecular switch that is temperature reactive (e.g., an aptamer that binds to, and deactivates, the polymerase enzyme at temperatures below 65° C., and/or unbinds, and activates, the polymerase enzyme at temperatures above 65° C.).

### Uracil-DNA Glycosylase

Aspects of the disclosure relate to solid compositions comprising a Uracil-DNA glycosylase (UDG) enzyme. UDG functions to eliminate uracil from DNA molecules by cleaving the N-glycosidic bond and, in cells, initiating the base-excision repair (BER) pathway. Without wishing to be bound by a particular theory, it is thought that the addition of dUTP and/or UDG during the amplification processes will reduce or eliminate potential contamination between samples. In the absence of adding dUTP and UDG, amplicons may aerosolize and contaminate future tests, potentially result in false positive test results. The use of UDG generally prevents carryover contamination by specifically degrading products have already been amplified (i.e., amplicons), leaving the unamplified (new) sample untouched and ready for amplification. Using this method, tests may be performed sequentially in the same tube and/or in the same area.

In some embodiments, a UDG enzyme is a recombinant UDG enzyme. In some embodiments, the recombinant UDG enzyme is expressed by bacteria, for example E. coli. In some embodiments, the recombinant UDG enzyme is derived from a psychrophilic marine bacterium (e.g., Psychrobacter sp.) or Sulphur metabolizing bacteria (e.g., Archaeoglobus fulgidus).

In some embodiments, a UDG enzyme is heat sensitive. A “heat sensitive” UDG enzyme generally refers to a UDG enzyme that ceases functioning (e.g., ceases to remove uracil from DNA) at temperatures above about 37° C., 40° C., 50° C., 60° C., or 65° C.

The concentration or amount of a UDG enzyme in a solid composition may vary. In some embodiments, the concentration of the UDG enzyme in a solid composition is at least 0.1 U/μL, at least 0.2 U/μL, at least 0.3 U/μL, at least 0.4 U/μL, at least 0.5 U/μL, at least 0.6 U/μL, at least 0.7 U/μL, at least 0.8 U/μL, at least 0.9 U/μL, or at least 1.0 U/μL. In some embodiments, the concentration of the UDG enzyme is in a range from 0.1 U/μL to 0.5 U/μL, 0.1 U/μL to 1.0 U/μL, 0.2 U/μL to 0.5 U/μL, 0.2 U/μL to 1.0 U/μL, or 0.5 U/μL to 1.0 U/μL. In some embodiments, the concentration of the UDG enzyme is at least 0.01 U/μL, at least 0.02 U/μL, at least 0.03 U/μL, at least 0.04 U/μL, or at least 0.05 U/μL. In certain embodiments, the concentration of the UDG enzyme is in a range from 0.01 U/μL to 0.02 U/μL, 0.01 U/μL to 0.03 U/μL, 0.01 U/μL to 0.04 U/μL, or 0.01 U/μL to 0.05 U/μL. In some embodiments, a “Unit” of UDG enzyme is defined as the amount of enzyme required to catalyzes the release of 60 μmol of uracil per minute from double-stranded, uracil-containing DNA. In some embodiments, UDG activity is measured by release of [3H]-uracil in a 50 μl reaction containing 0.2 μg DNA (104-105 cpm/m) in 30 minutes at 37° C.

In some embodiments, a UDG enzyme further comprises a molecular switch. In some embodiments, the molecular switch comprises one or more (e.g., 1, 2, 3, 4, 5, or more) binding site(s) for an agent that regulates activity of the UDG enzyme, for example an aptamer binding site, antibody binding site, small molecule binding site, etc. In some embodiments, the molecular switch controlling a UDG enzyme is reactive to environmental conditions, for example temperature, pH, salt concentration, etc. For example, in some embodiments, a solid composition comprises a heat sensitive UDG enzyme comprising a molecular switch that is temperature reactive (e.g., an aptamer that binds to, and deactivates, the UDG enzyme at temperatures above 37° C., and/or unbinds, and activates, the UDG enzyme at temperatures at or below 37° C.).

### Molecular Switches

Molecular switches, as used or described herein, are molecules (and their cognate binding partners) that, in response to certain conditions, reversibly switch between two or more stable states. In some embodiments, the condition that causes the molecular switch to change its configuration may be associated with any one or any combination of: pH, light, temperature, an electric current, microenvironment, and the presence of ions and/or other ligands. In some embodiments, the condition may be heat. In some embodiments, the molecular switches described herein comprise one or more aptamers. Aptamers generally refer to oligonucleotides or peptides that bind to specific target molecules (e.g., enzymes described herein, for example RT enzymes, polymerase enzymes, UDG enzymes, etc.). The aptamers, upon exposure to heat or other conditions, may dissociate from the enzymes. With the use of molecular switches, the processes described herein (e.g., lysis, decontamination, reverse transcription, and amplification, etc.) may be performed in a single test tube with a single enzymatic tablet, pellet, capsule, or gelcap.

Therefore, in some embodiments of the present technology, the molecular switches (e.g., aptamers) specifically bind the enzymes described herein, such that the enzymes are inactivated. The term “inactivated,” as used herein, may refer to or be used to describe an enzyme that is not enzymatically active; that is, it cannot perform its enzymatic function. Aptamers, as described herein, may be single-stranded nucleic acid molecules (about 5-25 kDa) having unique configurations that may allow them to bind to molecular targets with high specificity and affinity. In one embodiment, the aptamers may be DNA or RNA aptamers or hybrid DNA/RNA aptamers. Similar to antibodies, aptamers may possess binding affinities in the low nanomolar to picomolar range.

The small size of an aptamer may enhance its ability to bind to a specific site on an enzyme, thus enabling the aptamer to alter the function of that site without affecting the functions of other sites on the enzyme. In some embodiments of the present technology, the aptamers may inhibit the enzymatic activity of a reverse transcriptase, a DNA polymerase (e.g., Bst DNA polymerase), and/or a glycosylase. In some embodiments of the technology described herein, the presently disclosed methods may produce at least about a 10%, 15%, 20%, 25%, 30%, 35%, 40%, 45%, 50%, 55%, 60%, 65%, 70%, 75%, 80%, 85%, 90%, 95%, or even 100% inhibition of enzymatic activity relative to enzymatic activity measured in absence of aptamers (e.g., a control) in an assay.

The term “specifically binds,” as used herein, may refers to a molecule (e.g., an aptamer) that binds to a target (e.g., an enzyme) with at least five-fold greater affinity as compared to any non-targets, e.g., at least 10-, 20-, 50-, or 100-fold greater affinity. In some embodiments, a molecule (e.g., aptamer) that specifically binds to a target does not bind to any other non-target molecules.

The length of an aptamer may vary. In some embodiments an aptamer has a length of about 10 to about 120 nucleotides, such as about 15 nucleotides, about 20 nucleotides, about 25 nucleotides, about 30 nucleotides, about 35 nucleotides, about 40 nucleotides, about 45 nucleotides, about 50 nucleotides, about 55 nucleotides, about 60 nucleotides, about 65 nucleotides, about 70 nucleotides, about 75 nucleotides, about 80 nucleotides, about 85 nucleotides, about 90 nucleotides, about 95 nucleotides, about 100 nucleotides, about 105 nucleotides, about 110 nucleotides, about 115 nucleotides, about 120 nucleotides, or more nucleotides. In certain embodiments, the aptamer comprises one or more additional nucleotides attached to the 5′- and/or 3′ end of the aptamer.

The polynucleotide aptamers may be comprised of ribonucleotides only (RNA aptamers), deoxyribonucleotides only (DNA aptamers), or a combination of ribonucleotides and deoxyribonucleotides. The nucleotides may be naturally occurring nucleotides (e.g., ATP, TTP, GTP, CTP, UTP), or modified nucleotides, as described herein.

The molecular switch may be positioned on any part of the enzyme (e.g., UDG, RT enzyme, polymerase enzyme). In some embodiments, the molecular switch is positioned at or near the N-terminus of the enzyme (e.g., within about 1, 2, 3, 4, 5, 10, 20, or 50 amino acids of the N-terminus of the enzyme). In some embodiments, the molecular switch is positioned at or near the C-terminus of the enzyme (e.g., within about 1, 2, 3, 4, 5, 10, 20, or 50 amino acids of the C-terminus of the enzyme). In some embodiments, the molecular switch is positioned at, near, or within a catalytic or functional domain of the enzyme. In some embodiments, the molecular switch comprises the binding site on the enzyme and the agent (e.g., aptamer, small molecule, antibody, etc.) bound to the binding site. In some embodiments, the molecular switch refers only to the binding site (e.g., an aptamer binding site) on the enzyme.

### Solid Compositions

Aspects of the disclosure relate to solid compositions comprising two or more enzymes that may be controlled (e.g., sequentially or simultaneously activated and/or deactivated) by one or more molecular switches. In some embodiments, the solid compositions further comprise one or more pharmaceutically acceptable carriers or pharmaceutically acceptable excipients.

As used herein the term “pharmaceutically acceptable carrier” or “pharmaceutically acceptable excipient” refers to any and all solvents, dispersion media, coatings, antibacterial and antifungal agents, isotonic agents, and the like, compatible with use in diagnostic molecular assays. The use of such media and agents for pharmaceutically active substances is well known to in the art. Except insofar as any conventional media or agent is incompatible with the active compounds (e.g., reverse transcriptases, polymerases, etc.), use thereof in the compositions is contemplated. Supplementary active agents (e.g., additional enzymes, molecular switches, etc.) can also be incorporated into the compositions. in some embodiments, the solid compositions are sterile.

In some embodiments, a solid composition is in the form of a powder, granule, tablet, pellet, capsule, microcapsule, nanoparticle, microparticle, polymeric matrix, or gel. In some embodiments, one or more components of a solid composition has been freeze-dried or lyophilized prior to being included in the solid composition. In one example, a solid excipient may be formed by grinding a mixture (e.g., freeze dried or lyophilized enzyme or reaction mixture), and processing the mixture of granules, after adding suitable auxiliaries, if desired, to obtain tablets.

Examples of carriers or excipients used to formulate gel or polymeric solid compositions include but are not limited to calcium carbonate, calcium phosphate, various sugars, starches, cellulose derivatives, gelatin, and polymers such as polyethylene glycols.

In some embodiments, the solid composition is shelf stable for a relatively long period of time. In certain embodiments, the solid composition is shelf stable for at least 1 month, at least 3 months, at least 6 months, at least 9 months, at least 1 year, at least 2 years, at least 3 years, at least 4 years, at least 5 years, at least 6 years, at least 7 years, at least 8 years, at least 9 years, or at least 10 years. In some embodiments, the solid composition is shelf stable for 1-3 months, 1-6 months, 1-9 months, 1 month to 1 year, 1 month to 2 years, 1 month to 5 years, 1 month to 10 years, 3-6 months, 3-9 months, 3 months to 1 year, 3 months to 2 years, 3 months to 5 years, 3 months to 10 years, 6-9 months, 6 months to 1 year, 6 months to 2 years, 6 months to 5 years, 6 months to 10 years, 9 months to 1 year, 9 months to 2 years, 9 months to 5 years, 9 months to 10 years, 1-2 years, 1-3 years, 1-4 years, 1-5 years, 1-6 years, 1-7 years, 1-8 years, 1-9 years, 1-10 years, 2-5 years, 2-10 years, 3-5 years, 3-10 years, 4-10 years, 5-10 years, 6-10 years, 7-10 years, 8-10 years, or 9-10 years.

In some embodiments, the solid composition is thermostabilized and is stable across a wide range of temperatures. In some embodiments, the solid composition is stable at a temperature of at least 0° C., at least 10° C., at least 20° C., at least 37° C., at least 40° C., at least 50° C., at least 60° C., at least 65° C., at least 70° C., at least 80° C., at least 90° C., or at least 100° C. In some embodiments, the solid composition is stable at a temperature in a range from 0° C. to 10° C., 0° C. to 20° C., 0° C. to 37° C., 0° C. to 40° C., 0° C. to 50° C., 0° C. to 60° C., 0° C. to 65° C., 0° C. to 70° C., 0° C. to 80° C., 0° C. to 90° C., 0° C. to 100° C., 10° C. to 20° C., 10° C. to 37° C., 10° C. to 40° C., 10° C. to 50° C., 10° C. to 60° C., 10° C. to 65° C., 10° C. to 70° C., 10° C. to 80° C., 10° C. to 90° C., 10° C. to 100° C., 20° C. to 37° C., 20° C. to 40° C., 20° C. to 50° C., 20° C. to 60° C., 20° C. to 65° C., 20° C. to 70° C., 20° C. to 80° C., 20° C. to 90° C., 20° C. to 100° C., 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C.

Aspects of the disclosure relate to methods for producing a solid composition (e.g., an amplification tablet). In some embodiments, the method comprises obtaining one or more of a first enzyme (e.g., a reverse transcriptase) and a second enzyme (e.g., a polymerase), and optionally a third enzyme (e.g., a Uracil-DNA glycosylase), where each enzyme is under the control of a molecular switch, and compounding the enzymes to form a solid composition. Methods of compounding are known in the art and include direct compression, dry granulation, or wet granulation, for example as described in Kottke, M. and Rudnic, E. (2002), “Tablet Dosage Forms.” In G. Banker and C. Rhodes (Eds), Modern Pharmaceutics (pp. 437-511); New York: Marcel Dekker, Inc.

### Biological Samples

Aspects of the disclosure relate to methods for detecting one or more target nucleotides in a biological sample. In some embodiments, the biological sample is obtained from a subject (e.g., a human subject, an animal subject). Exemplary biological samples include bodily fluids (e.g. mucus, saliva, blood, serum, plasma, amniotic fluid, sputum, urine, cerebrospinal fluid, lymph, tear fluid, feces, or gastric fluid), cell scrapings (e.g., a scraping from the mouth or interior cheek), exhaled breath particles, tissue extracts, culture media (e.g., a liquid in which a cell, such as a pathogen cell, has been grown), environmental samples, agricultural products or other foodstuffs, and their extracts. In some embodiments, a biological sample comprises blood, saliva, mucus, urine, feces, cerebrospinal fluid (CSF), or tissue.

In some embodiments, the biological sample comprises a nasal secretion (e.g., mucus). In certain instances, for example, the sample is an anterior nares specimen. An anterior nares specimen may be collected from a subject by inserting a swab element of a sample-collecting component into one or both nostrils of the subject for a period of time. In some embodiments, the period of time is at least 5 seconds, at least 10 seconds, at least 20 seconds, or at least 30 seconds. In some embodiments, the period of time is 30 seconds or less, 20 seconds or less, 10 seconds or less, or 5 seconds or less. In some embodiments, the period of time is in a range from 5 seconds to 10 seconds, 5 seconds to 20 seconds, 5 seconds to 30 seconds, 10 seconds to 20 seconds, or 10 seconds to 30 seconds. In some embodiments, the biological sample comprises a cell scraping. In certain embodiments, the cell scraping is collected from the mouth or interior cheek. The cell scraping may be collected using a brush or scraping device formulated for this purpose.

In some embodiments, the sample comprises an oral secretion (e.g., saliva). In certain cases, the volume of saliva in the sample is at least 1 mL, at least 1.5 mL, at least 2 mL, at least 2.5 mL, at least 3 mL, at least 3.5 mL, or at least 4 mL. In some embodiments, the volume of saliva in the sample is in a range from 1 mL to 2 mL, 1 mL to 3 mL, 1 mL to 4 mL, or 2 mL to 4 mL. Saliva has been found to have a mean concentration of SARS-Cov-2 RNA of 5 fM (Kai-Wang To et al., 2020)—an amount that is detectable by any one of the methods described herein. In some embodiments, methods described herein are capable of detecting a concentration of a target nucleic acid (e.g., SARS-Cov-2 RNA) in a biological sample that is less than 5 fM.

The sample may be self-collected by the subject or may be collected by another individual (e.g., a family member, a friend, a coworker, a health care professional) using a sample-collecting component described herein.

In some embodiments, a biological sample (e.g., saliva sample) is deposited directly into a reaction tube. In some embodiments, the concentration of a target nucleic acid molecule (e.g., SARS-CoV-2 RNA) in the biological sample is at least 5 aM, at least 10 aM, at least 15 aM, at least 20 aM, at least 25 aM, at least 30 aM, at least 35 aM, at least 40 aM, at least 50 aM, at least 75 aM, at least 100 aM, at least 150 aM, at least 200 aM, at least 300 aM, at least 400 aM, at least 500 aM, at least 600 aM, at least 700 aM, at least 800 aM, at least 900 aM, at least 1 fM, at least 5 fM, at least 10 fM, at least 15 fM, at least 20 fM, at least 25 fM, at least 30 fM, at least 35 fM, at least 40 fM, at least 50 fM, at least 75 fM, at least 100 fM, at least 150 fM, at least 200 fM, at least 300 fM, at least 400 fM, at least 500 fM, at least 600 fM, at least 700 fM, at least 800 fM, at least 900 fM, at least 1 pM, at least 5 pM, or at least 10 pM. In some embodiments, the concentration of a target nucleic acid molecule (e.g., SARS-CoV-2 RNA) in the biological sample is 10 pM or less, 5 pM or less, 1 pM or less, 500 fM or less, 100 fM or less, 50 fM or less, 10 fM or less, 1 fM or less, 500 aM or less, 100 aM or less, 50 aM or less 10 aM or less, or 5 aM or less. In some embodiments, the concentration of a target nucleic acid molecule (e.g., SARS-CoV-2 RNA) in the biological sample is in a range from 5 aM to 50 aM, 5 aM to 100 aM, 5 aM to 500 aM, 5 aM to 1 fM, 5 aM to 10 fM, 5 aM to 50 fM, 5 aM to 100 fM, 5 aM to 500 fM, 5 aM to 1 pM, 5 aM to 10 pM, 10 aM to 50 aM, 10 aM to 100 aM, 10 aM to 500 aM, 10 aM to 1 fM, 10 aM to 10 fM, 10 aM to 50 fM, 10 aM to 100 fM, 10 aM to 500 fM, 10 aM to 1 pM, 10 aM to 10 pM, 100 aM to 500 aM, 100 aM to 1 fM, 100 aM to 10 fM, 100 aM to 50 fM, 100 aM to 100 fM, 100 aM to 500 fM, 100 aM to 1 pM, 100 aM to 10 pM, 1 fM to 10 fM, 1 fM to 50 fM, 1 fM to 100 fM, 1 fM to 500 fM, 1 fM to 1 pM, 1 fM to 10 pM, 5 fM to 10 fM, 5 fM to 50 fM, 5 fM to 100 fM, 5 fM to 500 fM, 5 fM to 1 pM, 5 fM to 10 pM, 10 fM to 100 fM, 10 fM to 500 fM, 10 fM to 1 pM, 10 fM to 10 pM, 100 fM to 500 fM, 100 fM to 1 pM, 100 fM to 10 pM, or 1 pM to 10 pM.

The biological sample, in some embodiments, is collected from a subject who is suspected of having the disease(s) the test screens for, such as a coronavirus (e.g., COVID-19) and/or influenza (e.g., influenza type A or influenza type B). Other indications, as described herein, are also envisioned. In some embodiments, the subject is a human. Subjects may be asymptomatic, or may present with one or more symptoms of the disease(s). Symptoms of coronaviruses (e.g., COVID-19) include, but are not limited to, fever, cough (e.g., dry cough), generalized fatigue, sore throat, headache, loss of taste or smell, runny nose, nasal congestion, muscle aches, and difficulty breathing (shortness of breath). Symptoms of influenza include, but are not limited to, fever, chills, muscle aches, cough, congestion, runny nose, headaches, and generalized fatigue. In some embodiments, the subject is asymptomatic, but has had contact within the past 14 days with a person that has tested positive for the virus.

A subject may be any mammal, for example a human, non-human primate (e.g., monkey, chimpanzee, ape, etc.), dog, cat, pig, horse, hamster, guinea pig, rat, mouse, etc. In some embodiments, a subject is a human. In some embodiments, a subject is an adult human (e.g., a human older than 16 years of age, 18 years of age, etc.). In some embodiments, a subject is a child (e.g., a pediatric subject), for example a subject that is less than 18 years of age, 16 years of age, etc. In some embodiments, a subject is an infant, for example a subject less than one year of age.

### Nucleic Acids

The disclosure relates, in some aspects, to nucleic acids and nucleic acid sequences. A “nucleic acid” sequence refers to a DNA or RNA (or a sequence encoded by DNA or RNA). In some embodiments, a nucleic acid is isolated. As used herein, the term “isolated” means artificially produced. As used herein, with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which can be manipulated by recombinant DNA techniques well known in the art. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art.

In some embodiments, a nucleic acid or isolated nucleic acid is a referred to as a “polynucleotide” or “oligonucleotide”. The terms “polynucleotide” and “oligonucleotide” refer to nucleic acids comprising two or more units (e.g., nucleotides) connected by a phosphate-based backbone (e.g., a sugar-phosphate backbone), for example genomic DNA (gDNA), complementary DNA (cDNA), RNA (e.g., mRNA, shRNA, dsRNA, miRNA, tRNA, etc.), synthetic nucleic acids and synthetic nucleic acid analogs. Polynucleotides (or oligonucleotides) may include natural or non-natural bases, or combinations thereof and natural or non-natural backbone linkages, such as phosphorothioate linkages, peptide nucleic acids (PNA), 2′-0-methyl-RNA, or combinations thereof.

The length of a polynucleotide (or each strand of a double stranded or duplex molecule) may vary. In some embodiments, a polynucleotide ranges from about 2 to 10, 2 to 20, 2 to 30, 2 to 40, 2 to 50, 2 to 75, 2 to 100, 2 to 150, 2 to 200, 2 to 300, 2 to 400, 2 to 500, 2 to 1000, 2 to 2000, 2 to 5000, 2 to 10,000, 2 to 50,000, 2 to 500,000, or 2 to 1,000,000 nucleotides in length. In some embodiments, a polynucleotide is more than 1,000,000 nucleotides in length (e.g., longer than a megabase).

A nucleic acid (e.g., a polynucleotide) may be single stranded or double stranded. In some embodiments, a single stranded polynucleotide comprises a sequence of polynucleotides connected by a contiguous backbone. In some embodiments, a single stranded polynucleotide comprises a 5′ portion (end or terminus) and a 3′ portion (end or terminus). A single stranded polynucleotide may be a sense strand or an antisense strand.

In some embodiments, a nucleic acid (e.g., polynucleotide) is double stranded. A double stranded polynucleotide comprises a first (e.g., “sense”) polynucleotide strand that is hybridized to a second polynucleotide (“antisense”) strand via hydrogen bonding between the nucleobases of each strand along a region of complementarity between the two strands. Each strand of a double stranded polynucleotide comprises a 5′ potion and a 3′ portion.

As used herein, the term “region of complementarity” refers to a region of a nucleic acid (e.g., polynucleotide) that is substantially complementary (e.g., forms Watson-Crick base pairs) to another nucleic acid sequence, for example a target nucleic acid sequence, control nucleic acid sequence, or a corresponding antisense strand (e.g., in the case of a double stranded or duplex nucleic acid). Two nucleic acids, for example a sense strand and antisense strand of a double stranded polynucleotide, may comprise a region of complementarity that ranges from about 70% to about 100% complementarity. In some embodiments, two polynucleotides share a region of complementarity that is about 50%, 60%, 70%, 80%, 90%, 95%, 99%, or 100% complementary. In some embodiments, a region of complementarity between two nucleic acids comprises at least 1, 2, 3, 4, 5, 6, 7, 8, 9, 10, 15, 20, 25, 50, 100, 200, or 500 mismatched nucleotide bases. In some embodiments, a region of complementarity between two nucleic acids comprises between 1 and 5, 2 and 10, 5 and 15, 10 and 50, 30 and 100, or 75 and 500 mismatched nucleotide bases. Mismatched nucleotide bases within a region of complementarity may result in the resulting complex comprising one or more bulges or loops.

In some embodiments, a single stranded polynucleotide or a double stranded polynucleotide forms a duplex region. A “duplex” refers to a stable complex formed by fully or partially complementary polynucleotides that undergo Watson-Crick type base pairing along a region of complementarity. For example, a sense strand and its reverse-complement antisense strand form a duplex molecule when hybridized (e.g., annealed) together. In another example, a single stranded polynucleotide forms a duplex molecule when two portions of the polynucleotide self-hybridize to form a stem-loop (e.g., hairpin) structure.

A duplex molecule may comprise one or two blunt ends. A “blunt end” refers to a double stranded polynucleotide (e.g., a duplex molecule) where the one end of the first (sense) polynucleotide strand and one end of the second (e.g., antisense) strand terminate at the same nucleotide position such that no overhang is formed. A blunt end may be formed at either the 5′ end or the 3′ end (e.g., with respect to the sense strand) of a duplex molecule. In some embodiments, a duplex molecule comprises two blunt ends. In some embodiments, a duplex molecule comprises one blunt end and one overhang. An “overhang” refers to a single stranded region of a duplex molecule formed by asymmetric hybridization of the first (e.g., sense) and second (e.g., antisense) strands of a duplex. Asymmetric hybridization may result from a difference in the lengths of two polynucleotides forming the duplex (e.g., one polynucleotide is longer than another) or from the two polynucleotide strands sharing a region of complementarity that does not encompass the entire length of one (or both) of the polynucleotides. Thus, in some embodiments, a duplex molecule comprises two overhangs.

The length of an overhang may vary. In some embodiments, an overhang ranges from between 1 and 100 nucleotides in length. In some embodiments, an overhang ranges from between 1 and 5 nucleotides in length. In some embodiments, an overhang ranges from between 2 and 10, 5 and 15, 10 and 25, or 20 and 100 nucleotides in length. In some embodiments, an overhang is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, a nucleic acid (e.g., polynucleotide) is a primer. As used herein, a “primer” refers to a polynucleotide that is capable of selectively binding (e.g., hybridizing or annealing) to a nucleic acid template and allows the synthesis of a sequence complementary to the corresponding polynucleotide template. A nucleic acid template may be a target nucleic acid or control nucleic acid. In some embodiments, a nucleic acid template comprises a region of complementarity with one or more primers. Generally, a primer ranges in size from about 10 to 100 nucleotides, and functions as a point of initiation for template-directed, polymerase mediated synthesis of a polynucleotide complementary to the template. In some embodiments, a primer is specific for a target nucleic acid. In some embodiments, a primer is specific for a control nucleic acid. A primer that is “specific” for a template binds (e.g., hybridizes) to that template with higher affinity than any other nucleic acid. In some embodiments, a primer that is “specific” for a template does not bind to any other nucleic acids present in a sample along with the template. A “pair of primers” or “primer pair” refers to two primers that bind to different regions (or portions) of the same template (e.g., the same target nucleic acid).

In some embodiments, a primer is an outer primer. An “outer primer” refers to a primer that binds at or near a 5′ end (e.g., at or near the 5′ terminal nucleotide) of a template sequence (e.g., a target nucleic acid, control nucleic acid, etc.), or at or near a 3′ end (e.g., at or near the 3′ terminal nucleotide) of a template sequence (e.g., a target nucleic acid, control nucleic acid, etc.) and is capable of displacing, or un-hybridizing, the two strands of a duplex upon hybridization of the primer to one of the duplex strands. In some embodiments, an outer primer binds to a terminal nucleotide, such as a 5′ terminal nucleotide or a 3′ terminal nucleotide of a template. In some embodiments, an outer primer binds within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides of the 5′ end (terminal nucleotide) of a template. In some embodiments, an outer primer binds within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides of the 3′ end (terminal nucleotide) of a template. In some embodiments, an outer primer is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, a primer is an internal primer. An “internal primer” refers to a primer that binds internally to an outer primer (e.g., downstream or 3′ relative to a 5′ outer primer, or upstream or 5′ relative to a 3′ outer primer). In some embodiments, internal primers are referred to as “nested primers”. In some embodiments, an internal primer binds to nucleic acid sequence that is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides of an outer primer. In some embodiments, an internal primer binds more than 50 nucleotides away from an outer primer, for example 60, 70, 80, 90, 100, 150, 200, or more nucleotides upstream or downstream of an outer primer. In some embodiments, an internal primer specifically hybridizes to a target nucleic acid. In some embodiments, an internal primer specifically hybridizes to a control nucleic acid. In some embodiments, an internal primer is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, an internal primer is a loop primer. A “loop primer” refers to a primer that binds to a single stranded duplex (e.g., loop) region of a template nucleic acid that is formed during Loop-mediated isothermal amplification (LAMP). In some embodiments, loop primers accelerate amplification (e.g., decrease the reaction time) during LAMP. In some embodiments, a loop primer binds to a region of a template (e.g., target nucleic acid) that is downstream of (e.g., 3′ relative to) an outer primer or another internal primer. In some embodiments, a loop primer binds to a region of a template (e.g., target nucleic acid) that is upstream of (e.g., 5′ relative to) an outer primer or another internal primer. In some embodiments, a loop primer is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

A nucleic acid may be unmodified or modified. A modified nucleotide may comprise one or more modified nucleic acid bases and/or a modified nucleic acid backbone. In some embodiments, a modified nucleic acid comprises one or more nucleotide analogs. The term “nucleotide analog” or “altered nucleotide” or “modified nucleotide” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function. Examples of positions of the nucleotide which may be derivatized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 August 10(4):297-310.

Nucleotide analogs may also comprise modifications to the sugar portion of the nucleotides. For example the 2′ OH-group may be replaced by a group selected from H, OR, R, F, Cl, Br, I, SH, SR, NH.sub.2, NHR, NR.sub.2, COOR, or, wherein R is substituted or unsubstituted C.sub.1-C.sub.6 alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 April 10(2):117-21, Rusckowski et al. Antisense Nucleic Acid Drug Dev. 2000 October 10(5):333-45, Stein, Antisense Nucleic Acid Drug Dev. 2001 Oct. 11(5): 317-25, Vorobjev et al. Antisense Nucleic Acid Drug Dev. 2001 April 11(2):77-85, and U.S. Pat. No. 5,684,143. Certain of the above-referenced modifications (e.g., phosphate group modifications) preferably decrease the rate of hydrolysis of, for example, polynucleotides comprising said analogs in vivo or in vitro.

In some embodiments, a nucleic acid is modified by conjugation to one or more biological or chemical moieties. Examples of moieties used for modifying nucleic acids include fluorophores, radioisotopes, chromophores, purification tags (e.g., polyHis, FLAG, biotin, etc.), barcoding molecules, haptens (e.g., FITC, digoxigenin (DIG), fluorescein, bovine serum albumin (BSA), dinitrophenyl, oxazole, pyrazole, thiazole, nitroaryl, benzofuran, triperpene, urea, thiourea, rotenoid, coumarin, etc.), extension blocking groups, and combinations thereof. In some embodiments, a nucleic acid (e.g., a primer) comprises one or more modifications. In some embodiments, a modified nucleic acid comprises a hapten. In some embodiments, the hapten is FITC. In some embodiments, the hapten is digoxigenin (DIG). In some embodiments, the hapten is biotin. In some embodiments, a modified nucleic acid comprises an extension blocking group. Examples of extension blocking groups include but are not limited to dehydroxylated 3′ nucleotide, 3′ dideoxycytosine (ddC), 3′ inverted deoxythymidine (dT), 3′ propyl (C3) spacer, 3′ amino modification, or 3′ phosphoryl modification. In some embodiments, a modified nucleic acid comprises both a hapten and an extension blocking group.

### Pathogens

Methods and compositions described by the disclosure may be used, in some embodiments, to detect the presence or absence of any target nucleic acid sequence (e.g., from any pathogen of interest). Target nucleic acid sequences may be associated with a variety of diseases or disorders, as described below. In some embodiments, the diagnostic devices, systems, and methods are used to diagnose at least one disease or disorder caused by a pathogen. In some embodiments, a disease or disorder caused by a pathogen is a sexually-transmitted infection (STI).

In certain instances, the diagnostic devices, systems, and methods are configured to detect a nucleic acid encoding a protein (e.g., a nucleocapsid protein) of SARS-CoV-2, which is the virus that causes COVID-19. In some embodiments, the diagnostic devices, systems, and methods are configured to identify particular strains of a pathogen (e.g., a virus). In certain embodiments, a diagnostic device comprises a lateral flow assay strip comprising a first test line configured to detect a nucleic acid sequence of SARS-CoV-2 and a second test line configured to detect a nucleic acid sequence of a SARS-CoV-2 virus having a D614G (i.e., a mutation of the 614^{th} amino acid from aspartic acid (D) to glycine (G)), N501Y, P681H, E484K, K417N, A701V, H655Y, L452R, T478K, and/or P681R mutation in its spike protein. In some embodiments, one or more target nucleic acid sequences are associated with a single-nucleotide polymorphism (SNP). In certain cases, diagnostic devices, systems, and methods described herein may be used for rapid genotyping to detect the presence or absence of a SNP, which may affect medical treatment.

In some embodiments, the diagnostic devices, systems, and methods are configured to diagnose two or more diseases or disorders. In certain cases, for example, a diagnostic device comprises a lateral flow assay strip comprising a first test line configured to detect a nucleic acid sequence of SARS-CoV-2 and a second test line configured to detect a nucleic acid sequence of an influenza virus (e.g., an influenza A virus or an influenza B virus). In some embodiments, a diagnostic device comprises a lateral flow assay strip comprising a first test line configured to detect a nucleic acid sequence of a virus and a second test line configured to detect a nucleic acid sequence of a bacterium. In some embodiments, a diagnostic device comprises a lateral flow assay strip comprising three or more test lines (e.g., test lines configured to detect SARS-CoV-2, SARS-CoV-2 D614G, an influenza type A virus, and/or an influenza type B virus). In some embodiments, a diagnostic device comprises a lateral flow assay strip comprising four or more test lines (e.g., test lines configured to detect SARS-CoV-2 viruses having one or more of the following mutations: D614G, N501Y, P681H, E484K, K417N, A701V, H655Y, L452R, L452Q, T478K, Q677H, S477N, R346K, F490S, Q414K, N450K, Ins214TDR, V367F, Q613H, A653V, P384L, S494P, N679K, Y449H, and/or P681R, an influenza type A virus, and/or an influenza type B virus).

In some embodiments, a diagnostic device, system, or method is configured to detect at least 1, at least 2, at least 3, at least 4, at least 5, at least 6, at least 7, at least 8, at least 9, or at least 10 target nucleic acid sequences. In some embodiments, the diagnostic device, system, or method is configured to detect 1 to 2 target nucleic acid sequences, 1 to 5 target nucleic acid sequences, 1 to 8 target nucleic acid sequences, 1 to 10 target nucleic acid sequences, 2 to 5 target nucleic acid sequences, 2 to 8 target nucleic acid sequences, 2 to 10 target nucleic acid sequences, 5 to 8 target nucleic acid sequences, 5 to 10 target nucleic acid sequences, or 8 to 10 target nucleic acid sequences. Each target nucleic acid sequence may independently be a nucleic acid of a pathogen (e.g., a viral, bacterial, fungal, protozoan, or parasitic pathogen) and/or a cancer cell.

In some embodiments, the diagnostic devices, systems, and methods are configured to detect a target nucleic acid sequence of a viral pathogen. Non-limiting examples of viral pathogens include coronaviruses, influenza viruses, rhinoviruses, parainfluenza viruses (e.g., parainfluenza 1-4), enteroviruses, adenoviruses, respiratory syncytial viruses, and metapneumoviruses. In certain embodiments, the viral pathogen is SARS-CoV-2 and/or SARS-CoV-2 D614G. In certain embodiments, the viral pathogen is an influenza virus. The influenza virus may be an influenza A virus (e.g., H1N1, H3N2) or an influenza B virus. In some embodiments, a diagnostic device, system, or method as described herein is configured to detect a target nucleic acid of a SARS-CoV-2 virus or a variant thereof, for example SARS-CoV-2 Alpha, SARS-CoV-2 Beta, SARS-CoV-2 Gamma, SARS-CoV-2 Delta, SARS-CoV-2 Eta, SARS-CoV-2 Theta, SARS-CoV-2 Kappa, SARS-CoV-2 Lambda, SARS-CoV-2 Iota, and/or SARS-CoV-2 Zeta. In some embodiments, a SARS-CoV-2 variant comprises one or more of the following spike protein mutations: D614G, N501Y, P681H, E484K, K417N, A701V, H655Y, L452R, L452Q, T478K, Q677H, S477N, R346K, F490S, Q414K, N450K, Ins214TDR, V367F, Q613H, A653V, P384L, S494P, N679K, Y449H, and/or P681R.

Other viral pathogens include, but are not limited to, adenovirus; Herpes simplex, type 1; Herpes simplex, type 2; encephalitis virus; papillomavirus (e.g., human papillomavirus); Varicella zoster virus; Epstein-Barr virus; human cytomegalovirus; human herpesvirus, type 8;

BK virus; JC virus; smallpox; polio virus; hepatitis A virus; hepatitis B virus; hepatitis C virus; hepatitis D virus; hepatitis E virus; human immunodeficiency virus (HIV); human bocavirus; parvovirus B19; human astrovirus; Norwalk virus; coxsackievirus; rhinovirus; Severe acute respiratory syndrome (SARS) virus; yellow fever virus; dengue virus; West Nile virus; Guanarito virus; Junin virus; Lassa virus; Machupo virus; Sabia virus; Crimean-Congo hemorrhagic fever virus; Ebola virus; Marburg virus; measles virus; mumps virus; rubella virus; Hendra virus; Nipah virus; Rabies virus; rotavirus; orbivirus; Coltivirus; Hantavirus; Middle East Respiratory Coronavirus; Zika virus; norovirus; Chikungunya virus; and Banna virus.

In some embodiments, the diagnostic devices, systems, and methods are configured to detect a target nucleic acid sequence of a bacterium (e.g., a bacterial pathogen). The bacterium may be a Gram-positive bacterium or a Gram-negative bacterium. Bacterial pathogens include, but are not limited to, Acinetobacter baumannii, Bacillus anthracis, Bacillus subtilis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, coagulase Negative Staphylococcus, Corynebacterium diphtheria, Enterococcus faecalis, Enterococcus faecium, Escherichia coli, enterotoxigenic Escherichia coli (ETEC), enteropathogenic E. coli, E. coli 0157:H7, Enterobacter sp., Francisella tularensis, Haemophilus influenzae, Helicobacter pylori, Klebsiella pneumoniae, Legionella pneumophila, Leptospira interrogans, Listeria monocytogenes, Moraxella catarralis, Mycobacterium leprae, Mycobacterium tuberculosis, Mycoplasma pneumoniae, Neisseria gonorrhoeae, Neisseria meningitides, Preteus mirabilis, Proteus sps., Pseudomonas aeruginosa, Rickettsia rickettsii, Salmonella typhi, Salmonella typhimurium, Serratia marcesens, Shigella flexneri, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, and Yersinia pestis.

In some embodiments, the diagnostic devices, systems, and methods are configured to detect a target nucleic acid sequence of a fungus (e.g., a fungal pathogen). Examples of fungal pathogens include, but are not limited to, Ascomycota (e.g., Fusarium oxysporum, Pneumocystis jirovecii, Aspergillus spp., Coccidioides immitis/posadasii, Candida albicans), Basidiomycota (e.g., Filobasidiella neoformans, Trichosporon), Microsporidia (e.g., Encephalitozoon cuniculi, Enterocytozoon bieneusi), and Mucoromycotina (e.g., Mucor circinelloides, Rhizopus oryzae, Lichtheimia corymbifera).

In some embodiments, the diagnostic devices, systems, and methods are configured to detect a target nucleic acid sequence of one or more protozoa (e.g., a protozoan pathogen).. Examples of protozoan pathogens include, but are not limited to, Entamoeba histolytica, Giardia lambila, Trichomonas vaginalis, Trypanosoma brucei, T cruzi, Leishmania donovani, Balantidium coli, Toxoplasma gondii, Plasmodium spp., and Babesia microti.

In some embodiments, the diagnostic devices, systems, and methods are configured to detect a target nucleic acid sequence of a parasite (e.g., a parasitic pathogen). Examples of parasitic pathogens include, but are not limited to, Acanthamoeba, Anisakis, Ascaris lumbricoides, botfly, Balantidium coli, bedbug, Cestoda, chiggers, Cochliomyia hominivorwc, Entamoeba histolytica, Fasciola hepatica, Giardia lamblia, hookworm, Leishmania, Linguatula serrata, Schistosoma (liver fluke), Loa, Paragonimus, pinworm, Plasmodium falciparum, Schistosoma, Strongyloides stercoralis, mite, tapeworm, Toxoplasma gondii, Trypanosoma, whipworm, and Wuchereria bancrofti.

In some embodiments, the diagnostic devices, systems, and methods are configured to detect a target nucleic acid sequence of a cancer cell. Cancer cells have unique mutations found in tumor cells and absent in normal cells. For example, the diagnostic devices, systems, and methods may be configured to detect a target nucleic acid sequence encoding a cancer neoantigen, a tumor-associated antigen (TAA), and/or a tumor-specific antigen (TSA). Examples of TAAs include, but are not limited to, MelanA (MART-I), gp100 (Pmel 17), tyrosinase, TRP-I, TRP-2, MAGE-I, MAGE-3, BAGE, GAGE-I, GAGE-2, p15(58), CEA, RAGE, NY-ESO (LAGE), SCP-I, HomNIe1-40, PRAME, p53, H-Ras, HER-2/neu, BCR-ABL, E2A-PRL, H4-RET, IGH-IGK, MYL- RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68VKP1, CO-029, FGF-5, G250, Ga733 (EpCAM), HTgp-175, M344, MA-50, MG7-Ag, MOV18, NB/70K, NY-CO-I, RCAS1, SDCCAG16, TA-90 (Mac-2 binding protein\cyclophilin C-associated protein), TAAL6, TAG72, TLP, and TPS5. Neoantigens, in some embodiments, arise from tumor proteins (e.g., tumor-associated antigens and/or tumor-specific antigens). In some embodiments, the neoantigen comprises a polypeptide comprising an amino acid sequence that is identical to a sequence of amino acids within a tumor antigen or oncoprotein (e.g., Her2, E7, tyrosinase-related protein 2 (Trp2), Myc, Ras, or vascular endothelial growth factor (VEGF)). In some embodiments, the amino acid sequence comprises at least 10, at least 15, at least 20, at least 25, at least 30, at least 35, at is least 40, at least 45, at least 50, at least 75, at least 100, at least 150, at least 200, or at least 250 amino acids. In some embodiments, the amino acid sequence comprises 10-250, 50-250, 100-250, or 50-150 amino acids.

In some embodiments, the diagnostic devices, systems, and methods are configured to examine a subject's predisposition to certain types of cancer based on specific genetic mutations.

As an example, mutations in BRCA1 and/or BRCA2 may indicate that a subject is at an increased risk of breast cancer, as compared to a subject who does not have mutations in the BRCA1 and/or BRCA2 genes. In some instances, the diagnostic devices, systems, and methods are configured to detect a target nucleic acid sequence comprising a mutation in BRCA1 and/or BRCA2. Other genetic mutations that may be screened according to the diagnostic devices, systems, and methods provided herein include, but are not limited to, BARD1, BRIP1, TP53, PTEN, MSH2, MLH1, MSH6, NF1, PMS1, PMS2, EPCAM, APC, RB1, MEN1, MEN2, and VHL. Further, determining a subject's genetic profile may help guide treatment decisions, as certain cancer drugs are indicated for subjects having specific genetic variants of particular cancers. For example, azathioprine, 6-mercaptopurine, and thioguanine all have dosing guidelines based on a subject's thiopurine methyltransferase (TPMT) genotype (see, e.g., The Pharmacogeneomics Knowledgebase, pharmgkb.org).

In some embodiments, the diagnostic devices, systems, and methods are configured to detect a target nucleic acid sequence associated with a genetic disorder. Non-limiting examples of genetic disorders include hemophilia, sickle cell anemia, α-thalassemia, β-thalassemia, Duchene muscular dystrophy (DMD), Huntington's disease, severe combined immunodeficiency, Marfan syndrome, hemochromatosis, and cystic fibrosis. In some embodiments, the target nucleic acid sequence is a portion of nucleic acid from a genomic locus of at least one of the following genes: CFTR, FMR1, SMN1, ABCB 11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASP A, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CUT A, CLN3, CLN5, CLN6, CLN8, CLRN1, CNGB3, COL27A1, COL4A3, COL4A4, COL4A5, COL7A1, CPS1, CPT1A, CPT2, CRB 1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHEL EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBAI, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAM A3, LAMB3, LAMC2, LCA5, LDLR, LDLRAP1, LHX3, LIFR, LIP A, LOXHD1, LPL, LRPPRC, MAN2B1, MCOLN1, MED 17, MESP2, 1VIF SD 8, MKS 1, MLC 1, MMAA, MMAB, MMACHC, MMADHC, MPI, MPL, MPV17, MTHFR, MTM1, MTRR, MTTP, MUT, MY07A, NAGLU, NAGS, NBN, NDRG1, NDUFAF5, NDUF S6, NEB, NPC1, NPC2, NPHS1, NPHS2, NR2E3, NTRK1, OAT, OP A3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, S1VIPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

In some embodiments, the diagnostic devices, systems, and methods are configured to detect a target nucleic acid sequence of an animal pathogen. Examples of animal pathogens include, but are not limited to, bovine rhinotracheitis virus, bovine herpesvirus, distemper, parainfluenza, canine adenovirus, rhinotracheitis virus, calicivirus, canine parvovirus, Borrelia burgdorferi (Lyme disease), Bordetella bronchiseptica (kennel cough), canine parainfluenza, leptospirosis, feline immunodeficiency virus, feline leukemia virus, Dirofilaria immitis (heartworm), feline herpesvirus, Chlamydia infections, Bordetella infections, equine influenza, rhinopneumonitis (equine herpesevirus), equine encephalomyelitis, West Nile virus (equine), Streptococcus equi, tetanus (Clostridium tetani), equine protozoal myeloencephalitis, bovine respiratory disease complex, clostridial disease, bovine respiratory syncytial virus, bovine viral diarrhea, Haemophilus somnus, Pasteurella haemolytica, and Pastuerella multocida.

The diagnostic devices, systems, and methods described herein may also be used to test water or food for contaminants (e.g., for the presence of one or more bacterial toxins). Bacterial contamination of food and water can result in foodborne diseases, which contribute to approximately 128,000 hospitalizations and 3000 deaths annually in the United States (CDC, 2016). In some cases, the diagnostic devices, systems, and methods described herein may be used to detect one or more toxins (e.g., bacterial toxins). In particular, bacterial toxins produced by Staphylococcus spp., Bacillus spp., and Clostridium spp. account for the majority of foodborne illnesses. Non-limiting examples of bacterial toxins include toxins produced by Clostridium botulinum, C. perfringens, Staphylococcus aureus, Bacillus cereus, Shiga-toxin-producing Escherichia coli (STEC), and Vibrio parahemolyticus . Exemplary toxins include, but are not limited to, aflatoxin, cholera toxin, diphtheria toxin, Salmonella toxin, Shiga toxin, Clostridium botulinum toxin, endotoxin, and mycotoxin. By testing a potentially contaminated food or water sample using the diagnostic devices, systems, or methods described herein, one can determine whether the sample contains the one or more bacterial toxins. In some embodiments, the diagnostic devices, systems, or methods may be operated or conducted during the food production process to ensure food safety prior to consumption.

In some embodiments, the diagnostic devices, systems, and methods described herein may be used to test samples of soil, building materials (e.g., drywall, ceiling tiles, wall board, fabrics, wallpaper, and floor coverings), air filters, environmental swabs, or any other sample. In certain embodiments, the diagnostic devices, systems, and methods may be used to detect one or more toxins, as described above. In certain instances, the diagnostic devices, systems, and methods may be used to analyze ammonia- and methane-oxidizing bacteria, fungi or other biological elements of a soil sample. Such information can be useful, for example, in predicting agricultural yields and in guiding crop planting decisions.

### Methods

In one illustrative embodiment of the present technology, an enzymatic tablet, pellet, capsule, or gelcap may comprise UDG, reverse transcriptase, and DNA polymerase (e.g., Bst DNA polymerase). Initially, the sample may be heated, for example at 37° C., which may be a temperature at which UDG is active, in order to decontaminate the sample. At 37° C., molecular switches may bind to, and inactivate, the reverse transcriptase and DNA polymerase. This may advantageously ensure that they do not interfere with the UDG decontamination reaction. Next, following decontamination, the sample may be heated, for example at 65° C., which may deactivate heat-sensitive UDG but may cause the molecular switches to release, and therefore activate, the reverse transcriptase and DNA polymerase. Reverse transcription, and subsequent amplification (e.g., by LAMP) may then proceed.

A biological sample may be lysed prior to (or while) performing an amplification reaction (e.g., an isothermal amplification reaction, such as RT-LAMP). In some embodiments, lysis of a biological sample is performed by chemical lysis (e.g., exposing a sample to one or more lysis reagents) and/or thermal lysis (e.g., heating a sample). Chemical lysis may be performed by one or more lysis reagents. In some embodiments, the one or more lysis reagents comprise one or more enzymes. Non-limiting examples of suitable enzymes include lysozyme, lysostaphin, zymolase, cellulase, protease, and glycanase.

In some embodiments, the one or more lysis reagents comprise one or more detergents. Non-limiting examples of suitable detergents include sodium dodecyl sulphate (SDS), Tween (e.g., Tween 20, Tween 80), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and NP-40. In some embodiments, a composition (e.g., a composition comprising a solid composition and one or more buffers) described herein comprises one or more detergents. In some embodiments, the one or more detergents comprises Tween.

In some cases, at least one of the one or more lysis reagents is in solid form (e.g., lyophilized, dried, crystallized, air jetted). In some cases, all of the one or more lysis reagents are in solid form (e.g., lyophilized, dried, crystallized, air jetted). In certain embodiments, one or more lysis reagents are in the form of a lysis pellet or tablet. The lysis pellet or tablet may comprise any lysis reagent described herein.

In certain embodiments, the lysis pellet or tablet may comprise one or more additional reagents (e.g., reagents to reduce or eliminate cross contamination). In a particular, non-limiting embodiment, a lysis pellet or tablet comprises Thermolabile Uracil-DNA Glycosylase (UDG) (e.g., at a concentration of about 0.02 U/μL) and murine RNAse inhibitor (e.g., at a concentration of about 1 U/μL). In some embodiments, the lysis pellet or tablet further comprises a reverse transcriptase enzyme (e.g., a reverse transcriptase enzyme comprising a molecular switch) and/or a polymerase enzyme (e.g., a polymerase enzyme comprising a molecular switch).

In some embodiments, the lysis pellet or tablet is shelf stable for a relatively long period of time. In certain embodiments, the lysis pellet or tablet is shelf stable for at least 1 month, at least 3 months, at least 6 months, at least 9 months, at least 1 year, at least 2 years, at least 3 years, at least 4 years, at least 5 years, at least 6 years, at least 7 years, at least 8 years, at least 9 years, or at least 10 years. In some embodiments, the lysis pellet or tablet is shelf stable for 1-3 months, 1-6 months, 1-9 months, 1 month to 1 year, 1 month to 2 years, 1 month to 5 years, 1 month to 10 years, 3-6 months, 3-9 months, 3 months to 1 year, 3 months to 2 years, 3 months to 5 years, 3 months to 10 years, 6-9 months, 6 months to 1 year, 6 months to 2 years, 6 months to 5 years, 6 months to 10 years, 9 months to 1 year, 9 months to 2 years, 9 months to 5 years, 9 months to 10 years, 1-2 years, 1-3 years, 1-4 years, 1-5 years, 1-6 years, 1-7 years, 1-8 years, 1-9 years, 1-10 years, 2-5 years, 2-10 years, 3-5 years, 3-10 years, 4-10 years, 5-10 years, 6-10 years, 7-10 years, 8-10 years, or 9-10 years.

In some embodiments, the lysis pellet or tablet is thermostabilized and is stable across a wide range of temperatures. In some embodiments, the lysis pellet or tablet is stable at a temperature of at least 0° C., at least 10° C., at least 20° C., at least 37° C., at least 40° C., at least 50° C., at least 60° C., at least 65° C., at least 70° C., at least 80° C., at least 90° C., or at least 100° C. In some embodiments, the lysis pellet or tablet is stable at a temperature in a range from 0° C. to 10° C., 0° C. to 20° C., 0° C. to 37° C., 0° C. to 40° C., 0° C. to 50° C., 0° C. to 60° C., 0° C. to 65° C., 0° C. to 70° C., 0° C. to 80° C., 0° C. to 90° C., 0° C. to 100° C., 10° C. to 20° C., 10° C. to 37° C., 10° C. to 40° C., 10° C. to 50° C., 10° C. to 60° C., 10° C. to 65° C., 10° C. to 70° C., 10° C. to 80° C., 10° C. to 90° C., 10° C. to 100° C., 20° C. to 37° C., 20° C. to 40° C., 20° C. to 50° C., 20° C. to 60° C., 20° C. to 65° C., 20° C. to 70° C., 20° C. to 80° C., 20° C. to 90° C., 20° C. to 100° C., 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C.

In some embodiments, the one or more lysis reagents are active at approximately room temperature (e.g., 20° C.-25° C.). In some embodiments, the one or more lysis reagents are active at elevated temperatures (e.g., at least 37° C., at least 40° C., at least 50° C., at least 60° C., at least 65° C., at least 70° C., at least 80° C., at least 90° C.). In some embodiments, chemical lysis is performed at a temperature in a range from 20° C. to 25° C., 20° C. to 30° C., 20° C. to 37° C., 20° C. to 50° C., 20° C. to 60° C., 20° C. to 65° C., 20° C. to 70° C., 20° C. to 80° C., 20° C. to 90° C., 25° C. to 30° C., 25° C. to 37° C., 25° C. to 50° C., 25° C. to 60° C., 25° C. to 65° C., 25° C. to 70° C., 25° C. to 80° C., 25° C. to 90° C., 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C.

In some embodiments, cell lysis is accomplished by applying heat to a sample (thermal lysis). In certain instances, thermal lysis is performed by applying a lysis heating protocol comprising heating the sample at one or more temperatures for one or more time periods using any heater described herein. In some embodiments, a lysis heating protocol comprises heating the sample at a first temperature for a first time period. In certain instances, the first temperature is at least 37° C., at least 50° C., at least 60° C., at least 65° C., at least 70° C., at least 80° C., or at least 90° C. In certain instances, the first temperature is in a range from 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the first time period is at least 1 minute, at least 2 minutes, at least 3 minutes, at least 4 minutes, at least 5 minutes, at least 10 minutes, at least 15 minutes, at least 20 minutes, or at least 30 minutes. In certain instances, the first time period is in a range from 1 to 3 minutes, 1 to 5 minutes, 1 to 10 minutes, 1 to 15 minutes, 1 to 20 minutes, 1 to 30 minutes, 3 to 5 minutes, 3 to 10 minutes, 3 to 15 minutes, 3 to 20 minutes, 3 to 30 minutes, 5 to 10 minutes, 5 to 15 minutes, 5 to 20 minutes, 5 to 30 minutes, 10 to 20 minutes, 10 to 30 minutes, or 20 to 30 minutes. In some embodiments, a lysis heating protocol comprises heating the sample at a second temperature for a second time period. In certain instances, the second temperature is at least 37° C., at least 50° C., at least 60° C., 9512067.1at least 65° C., at least 70° C., at least 80° C., or at least 90° C. In certain instances, the second temperature is in a range from 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the second time period is at least 1 minute, at least 2 minutes, at least 3 minutes, at least 4 minutes, at least 5 minutes, at least 10 minutes, at least 15 minutes, at least 20 minutes, or at least 30 minutes. In certain instances, the second time period is in a range from 1 to 3 minutes, 1 to 5 minutes, 1 to 10 minutes, 1 to 15 minutes, 1 to 20 minutes, 1 to 30 minutes, 3 to 5 minutes, 3 to 10 minutes, 3 to 15 minutes, 3 to 20 minutes, 3 to 30 minutes, 5 to 10 minutes, 5 to 15 minutes, 5 to 20 minutes, 5 to 30 minutes, 10 to 20 minutes, 10 to 30 minutes, or 20 to 30 minutes. In a particular, non-limiting embodiment, the first temperature is in a range from 37° C. to 50° C. (e.g., about 37° C.) and the first time period is in a range from 1 minute to 5 minutes (e.g., about 3 minutes), and the second temperature is in a range from 60° C. to 70° C. (e.g., about 65° C.) and the second time period is in a range from 5 minutes to 15 minutes (e.g., about 10 minutes). In some embodiments, a lysis heating protocol may comprise heating a sample at one or more additional temperatures for one or more additional time periods.

In some embodiments, DNA may be amplified according to any nucleic acid amplification method known in the art. In some embodiments, the nucleic acid amplification method is an isothermal amplification method. Isothermal amplification methods include, but are not limited to, loop-mediated isothermal amplification (LAMP), recombinase polymerase amplification (RPA), nicking enzyme amplification reaction (NEAR), nucleic acid sequence-based amplification (NASBA), strand displacement amplification (SDA), helicase-dependent amplification (HDA), isothermal multiple displacement amplification (IMDA), rolling circle amplification (RCA), transcription mediated amplification (TMA), signal mediated amplification of RNA technology (SMART), single primer isothermal amplification (SPIA), circular helicase-dependent amplification (cHDA), and whole genome amplification (WGA). In one embodiment, the nucleic acid amplification method is loop-mediated isothermal amplification (LAMP). In another embodiment, the nucleic acid amplification method is recombinase polymerase amplification (RPA). In another embodiment, the nucleic acid amplification method is nicking enzyme amplification reaction.

In some embodiments, the isothermal amplification methods described below include a modified nucleotide, for example, deoxyuridine triphosphate (dUTP), during amplification. In such embodiments, a subsequent test may comprise a uracil-DNA glycosylase (UDG) treatment prior to the amplification step, followed by a heat inactivation step (e.g., 95° C. for 5 minutes) (Hsieh et al., Chem Comm, 2014, 50: 3747-3749). In some embodiments, the heat inactivation step may correspond to a thermal lysis step.

In some cases, at least one of the one or more amplification reagents is in solid form (e.g., lyophilized, dried, crystallized, air jetted). In some cases, all of the one or more amplification reagents are in solid form (e.g., lyophilized, dried, crystallized, air jetted). In certain embodiments, one or more amplification reagents are in the form of an amplification pellet or tablet. The amplification pellet or tablet may comprise any amplification reagent described herein.

In some embodiments, the nucleic acid amplification reagents contained in the solid composition are LAMP reagents. LAMP refers to a method of amplifying a target nucleic acid using at least four primers through the creation of a series of stem-loop structures. Due to its use of multiple primers, LAMP may be highly specific for a target nucleic acid sequence. In some embodiments, LAMP is combined with a reverse transcription reaction, and referred to as RT-LAMP.

In some embodiments, the LAMP reagents comprise four or more primers. In certain embodiments, the four or more primers comprise a forward inner primer (FIP), a backward inner primer (BIP), a forward outer primer (F3), and a backward outer primer (B3). In some cases, the four or more primers target at least six specific regions of a target gene. In some embodiments, the LAMP reagents further comprise a forward loop primer (Loop F or LF) and a backward loop primer (Loop B or LB). In certain cases, the loop primers target cyclic structures formed during amplification and can accelerate amplification.

Methods of designing LAMP primers are known in the art. In some cases, LAMP primers may be designed for each target nucleic acid a diagnostic device is configured to detect. For example, a diagnostic device configured to detect a first target nucleic acid (e.g., a nucleic acid of SARS-CoV-2) and a second target nucleic acid (e.g., a nucleic acid of an influenza virus) may comprise a first set of LAMP primers directed to the first target nucleic acid and a second set of LAMP primers directed to the second target nucleic acid. In some embodiments, the LAMP primers may be designed by alignment and identification of conserved sequences in a target pathogen (e.g., using Clustal X or a similar program) and then using a software program (e.g., PrimerExplorer). The specificity of different candidate primers may be confirmed using a BLAST search of the GenBank nucleotide database. Primers may be synthesized using any method known in the art.

In certain embodiments, the target pathogen is SARS-CoV-2. In some cases, primers for amplification of a SARS-CoV-2 nucleic acid sequence are selected from regions of the virus's nucleocapsid (N) gene, envelope (E) gene, membrane (M) gene, and/or spike (S) gene. In some instances, primers were selected from regions of the SARS-CoV-2 nucleocapsid (N) gene to maximize inclusivity across known SARS-CoV-2 strains and minimize cross-reactivity with related viruses and genomes that may be presence in the sample.

In some embodiments, the LAMP reagents comprise deoxyribonucleotide triphosphates (“dNTPs”). In certain embodiments, the LAMP reagents comprise deoxyadenosine triphosphate (“dATP”), deoxyguanosine triphosphate (“dGTP”), deoxycytidine triphosphate (“dCTP”), and deoxythymidine triphosphate (“dTTP”). In certain embodiments, the concentration of each dNTP (i.e., dATP, dGTP, dCTP, dTTP) is at least 0.5 mM, at least 0.6 mM, at least 0.7 mM, at least 0.8 mM, at least 0.9 mM, at least 1.0 mM, at least 1.1 mM, at least 1.2 mM, at least 1.3 mM, at least 1.4 mM, at least 1.5 mM, at least 1.6 mM, at least 1.7 mM, at least 1.8 mM, at least 1.9 mM, or at least 2.0 mM. In some embodiments, the concentration of each dNTP is in a range from 0.5 mM to 1.0 mM, 0.5 mM to 1.5 mM, 0.5 mM to 2.0 mM, 1.0 mM to 1.5 mM, 1.0 mM to 2.0 mM, or 1.5 mM to 2.0 mM.

In some embodiments, the LAMP reagents comprise magnesium sulfate (MgSO₄). In certain embodiments, the concentration of MgSO₄ is at least 1 mM, at least 2 mM, at least 3 mM, at least 4 mM, at least 5 mM, at least 6 mM, at least 7 mM, at least 8 mM, at least 9 mM, or at least 10 mM. In certain embodiments, the concentration of MgSO4 is in a range from 1 mM to 2 mM, 1 mM to 5 mM, 1 mM to 8 mM, 1 mM to 10 mM, 2 mM to 5 mM, 2 mM to 8 mM, 2 mM to 10 mM, 5 mM to 8 mM, 5 mM to 10 mM, or 8 mM to 10 mM.

In some embodiments, the LAMP reagents comprise betaine. In certain embodiments, the concentration of betaine is at least 0.1 M, at least 0.2 M, at least 0.3 M, at least 0.4 M, at least 0.5 M, at least 0.6 M, at least 0.7 M, at least 0.8 M, at least 0.9 M, at least 1.0 M, at least 1.1 M, at least 1.2 M, at least 1.3 M, at least 1.4 M, or at least 1.5 M. In certain embodiments, the concentration of betaine is in a range from 0.1 M to 0.2 M, 0.1 M to 0.5 M, 0.1 M to 0.8 M, 0.1 M to 1.0 M, 0.1 M to 1.2 M, 0.1 M to 1.5 M, 0.2 M to 0.5 M, 0.2 M to 0.8 M, 0.2 M to 1.0 M, 0.2 M to 1.2 M, 0.2 M to 1.5 M, 0.5 M to 0.8 M, 0.5 M to 1.0 M, 0.5 M to 1.2 M, 0.5 M to 1.5 M, 0.8 M to 1.0 M, 0.8 M to 1.2 M, 0.8 M to 1.5 M, 1.0 M to 1.2 M, or 1.0 M to 1.5 M.

In some embodiments, amplified nucleic acids (i.e., amplicons of target nucleic acids or control nucleic acids) may be detected using any suitable method. In some embodiments, one or more target nucleic acid sequences are detected using a lateral flow assay strip. In some embodiments, one or more target nucleic acid sequences are detected using a colorimetric assay. In some embodiments, the disclosure relates to rapid, self-administrable tests for detecting the presence of one or more target nucleic acids derived from one or more pathogens. A “self-administrable” test refers to a test in which all testing steps are performed by the subject of the test. In some embodiments, a self-administrable test is performed by a subject at a location that is not a medical facility (e.g., a hospital, physicians' office, nurse's office, etc.). In some embodiments, a self-administrable test is performed at the subject's home. In some embodiments, a detection of one or more pathogens using a method described herein is performed at a point of care, for example at a hospital. In some embodiments, a detection of one or more pathogens using a method described herein is performed by a healthcare professional (e.g., doctor, nurse, physician assistant, laboratory technician, etc.) on a biological sample obtained from a subject. In some embodiments, a “rapid test” refers to a test in which all testing steps (e.g., sample collection, lysis, isothermal amplification, detection, etc.) may be completed in less than 3 hours, less than 2 hours, or less than 1 hour. In some embodiments, a rapid test is completed (e.g., one of more nucleic acids are detected) in less than 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or 5 minutes.

### Diagnostic Systems

Compositions and methods described herein may be part of a diagnostic system. For example, a diagnostic system may comprise one or more reagents, and at least one of the one or more reagents may be a solid composition as described herein. In some embodiments, one or more solid composition is contained in a container (e.g., reaction tube) that is part of a diagnostic system described herein. In some embodiments, a diagnostic system as described herein is used to carry out a method of lysing, amplifying, and detecting one or more pathogens in a biological sample using a solid composition described by the disclosure.

As the COVID-19 pandemic has highlighted, there is a critical need for rapid, accurate systems and methods for diagnosing diseases—particularly infectious diseases. In the absence of diagnostic testing, asymptomatic infected individuals may unknowingly spread the disease to others, and symptomatic infected individuals may not receive appropriate treatment. With testing, however, infected individuals may take appropriate precautions (e.g., self-quarantine) to reduce the risk of infecting others and may receive targeted treatment as helpful.

While diagnostic tests for various diseases, including COVID-19, are known, such tests often require specialized knowledge of laboratory techniques and/or expensive laboratory equipment. For example, polymerase chain reaction (PCR) tests generally require skilled technicians and expensive, bulky thermocyclers. In addition, there is a need for diagnostic tests that are both rapid and highly accurate. Known diagnostic tests with high levels of accuracy often take hours, or even days, to return results, and more rapid tests generally have low levels of accuracy. Many rapid diagnostic tests detect antibodies, which generally can only reveal whether a person has previously had a disease, not whether the person has an active infection. In contrast, nucleic acid tests (i.e., tests that detect one or more target nucleic acid sequences) may indicate that a person has an active infection.

Diagnostic devices, systems, and methods comprising solid compositions described herein may be safely and easily operated or conducted by untrained individuals. Unlike prior art diagnostic tests, some embodiments described herein may not require knowledge of even basic laboratory techniques (e.g., pipetting). Similarly, some embodiments described herein may not require expensive laboratory equipment (e.g., thermocyclers). In some embodiments, reagents are contained within a reaction tube, a cartridge, and/or a blister pack, such that users are not exposed to any potentially harmful chemicals.

Diagnostic devices, systems, and methods described herein are also highly sensitive and accurate. In some embodiments, the diagnostic devices, systems, and methods are configured to detect one or more target nucleic acid sequences using nucleic acid amplification (e.g., an isothermal nucleic acid amplification method). Through nucleic acid amplification, the diagnostic devices, systems, and methods are able to accurately detect the presence of extremely small amounts of a target nucleic acid. In certain cases, for example, the diagnostic devices, systems, and methods can detect 1 pM or less, or 10 aM or less.

As a result, the diagnostic devices, systems, and methods described herein may be useful in a wide variety of contexts. For example, in some cases, the diagnostic devices and systems may be available over the counter for use by consumers. In such cases, untrained consumers may be able to self-administer the diagnostic test (or administer the test to friends and family members) in their own homes (or any other location of their choosing). In some cases, the diagnostic devices, systems, or methods may be operated or performed by employees or volunteers of an organization (e.g., a school, a medical office, a business). For example, a school (e.g., an elementary school, a high school, a university) may test its students, teachers, and/or administrators, a medical office (e.g., a doctor's office, a dentist's office) may test its patients, or a business may test its employees for a particular disease. In each case, the diagnostic devices, systems, or methods may be operated or performed by the test subjects (e.g., students, teachers, patients, employees) or by designated individuals (e.g., a school nurse, a teacher, a school administrator, a receptionist).

In some embodiments, diagnostic devices described herein are relatively small. In certain cases, for example, a device is approximately the size of a pen or a marker. Thus, unlike diagnostic tests that require bulky equipment, diagnostic devices and systems described herein may be easily transported and/or easily stored in homes and businesses. In some embodiments, the diagnostic devices and systems are relatively inexpensive. Since no expensive laboratory equipment (e.g., a thermocycler) is required, diagnostic devices, systems, and methods described herein may be more cost effective than known diagnostic tests. In some embodiments, any reagents contained within a diagnostic device or system described herein may be thermostabilized, and the diagnostic device or system may be shelf stable for a relatively long period of time. In certain embodiments, for example, the diagnostic device or system may be stored at room temperature (e.g., 20° C. to 25° C.) for a relatively long period of time (e.g., at least 1 month, at least 3 months, at least 6 months, at least 9 months, at least 1 year, at least 5 years, at least 10 years). In certain embodiments, the diagnostic device or system may be stored across a range of temperatures (e.g., 0° C. to 20° C., 0° C. to 37° C., 0° C. to 60° C., 0° C. to 90° C., 20° C. to 37° C., 20° C. to 60° C., 20° C. to 90° C., 37° C. to 60° C., 37° C. to 90° C., 60° C. to 90° C.) for a relatively long period of time (e.g., at least 1 month, at least 3 months, at least 6 months, at least 9 months, at least 1 year, at least 5 years, at least 10 years).

The present disclosure provides diagnostic devices, systems, and methods for rapidly and in a home environment detecting one or more target nucleic acid sequences (e.g., a nucleic acid sequence of a pathogen, such as SARS-CoV-2 or an influenza virus). A diagnostic system, as described herein, may be self-administrable and comprise a sample-collecting component (e.g., a swab) and a diagnostic device. The diagnostic device may comprise a cartridge, a blister pack, and/or a “chimney” detection device, according to some embodiments. In some cases, the diagnostic device comprises a detection component (e.g., a lateral flow assay strip, a colorimetric assay), results of which are self-readable, or automatically read by a computer algorithm. In certain embodiments, the diagnostic device further comprises one or more reagents (e.g., lysis reagents, nucleic acid amplification reagents, CRISPR/Cas detection reagents). In certain other embodiments, the diagnostic system separately includes one or more reaction tubes comprising the one or more reagents. The diagnostic device may also comprise an integrated heater, or the diagnostic system may comprise a separate heater. The isothermal amplification technique employed yields not only fast but very accurate results.

In some embodiments, at least one reagent is not contained within a diagnostic device, and a diagnostic system comprises one or more reaction tubes. The one or more reaction tubes may contain any reagent(s) described above. In some embodiments, the one or more reaction tubes comprise at least one reagent in liquid form. In some embodiments, the one or more reaction tubes comprise at least one reagent in solid form.

A reaction tube of a diagnostic system may be formed from any suitable material. In some embodiments, the reaction tube is formed from a polymer. Non-limiting examples of suitable polymers include polypropylene (PP), polytetrafluoroethylene (PTFE), polyurethane (PU), polyvinyl chloride (PVC), polystyrene, neoprene, nitrile, nylon and polyamide. In some embodiments, the reaction tube comprises glass and/or a ceramic. The glass may, in some instances, be an expansion-resistant glass (e.g., borosilicate glass or fused quartz). In some embodiments, the reaction tube is an Eppendorf tube. In some embodiments, the reaction tube has a substantially flat bottom (e.g., the reaction tube can stand on its own), a substantially round bottom, or a substantially conical bottom. If the reaction tube has a round or conical bottom, or any other bottom that does not allow the reaction tube to readily stand on its own, the diagnostic system may further comprise a stand for the reaction tube. In some embodiments, the reaction tube is sterile.

The reaction tubes, in some embodiments, further comprise at least one cap. In some embodiments, the reaction tube comprises a partially removable cap (e.g., a hinged cap) or one or more wholly removable caps (e.g., one or more screw-top caps, one or more stoppers). In some embodiments, the one or more caps comprise reagents in solid form (e.g., lyophilized, dried, crystallized, air jetted reagents).

The diagnostic system, in some embodiments, comprises a heater. In certain embodiments, the heater is integrated with the diagnostic device. In some instances, for example, the heater is a printed circuit board (PCB) heater. The PCB heater, in some embodiments, comprises a bonded PCB with a microcontroller, thermistors, and/or resistive heaters. In certain embodiments, the diagnostic device comprises a cartridge and/or a blister pack comprising one or more reservoirs (e.g., a lysis reservoir, a nucleic acid amplification reservoir). In some embodiments, the PCB heater is in thermal communication with at least one of the one or more reservoirs. In some embodiments, the PCB heater is located adjacent to (e.g., below) at least one of the one or more reservoirs. amplification reservoirs) In some embodiments, the diagnostic system comprises a separate heater (i.e., a heater that is not integrated with other system components). In some cases, the heater comprises a battery-powered heat source, a USB-powered heat source, a hot plate, a heating coil, and/or a hot water bath. In certain embodiments, the heating unit is contained within a thermally-insulated housing to ensure user safety. In certain instances, the heating unit is an off-the-shelf consumer-grade device. In some embodiments, the heat source is a thermocycler or other specialized laboratory equipment known in the art. In some embodiments, the heater is configured to receive a reaction tube.

In some embodiments, a diagnostic system comprises instructions associated with system components. The instructions may include instructions for performing any one of the diagnostic methods provided herein. The instructions may include instructions for the use, modification, mixing, diluting, preserving, administering, assembly, storage, packaging, and/or preparation of system components. The instructions may be provided in any form recognizable by one of ordinary skill in the art as a suitable vehicle for containing such instructions. For example, the instructions may be written or published, verbal, audible (e.g., telephonic), digital, optical, visual (e.g., videotape, DVD, etc.), and/or provided via electronic communications (including Internet or web-based communications). In some embodiments, the instructions are provided as part of a software-based application, as described herein.

In some embodiments, one or more components of a diagnostic system comprise a unique label. In some cases, this may advantageously allow multiple samples to be run in parallel. For example, one or more components of the diagnostic system (e.g., reaction tube cap, detection component) may be labeled with the same label. In some embodiments, a copy of the label is given to a tested subject, so that the subject may later receive the results using the unique label. In this way, multiple tests (one for each unique subject) may be run in parallel without mixing up the samples.

### EXAMPLE

This example describes one embodiment of a diagnostic method described by the disclosure. A biological sample (e.g., saliva) is obtained from a subject. The biological sample is lysed to release nucleic acids from cells. The lysate is contacted to a solid composition comprising 1) a heat sensitive UDG enzyme, 2) a heat stable reverse transcriptase (RT) enzyme comprising a molecular switch, and 3) a low temp inactive polymerase enzyme comprising a molecular switch, to produce a reaction mixture.

The reaction mixture is heated to 37° C. At 37° C., several processes occur—the UDG is active and functions to remove contamination from the lysate; and the aptamer portions of the molecular switches for RT enzyme and polymerase enzyme are bound to their cognate binding sites on the enzymes, rendering the enzymes inactive.

Once decontamination is complete, the reaction mixture is then heated to 65° C. At 65° C., several processes occur—the heat sensitive UDG is inactivated; and the aptamer-portions of the molecular switches for the RT enzyme and polymerase enzyme dissociate from their cognate binding sites and activate the enzymes.

Next, isothermal amplification of the nucleic acids (e.g., by LAMP) proceeds. The LAMP amplicons are then detected.

1. A solid composition comprising:
(i) a first enzyme; and
(ii) a second enzyme,
wherein the first enzyme and second enzyme are each under the control of a molecular switch.

2. The solid composition of claim 1, wherein the composition is in the form of a pellet, capsule, gelcap, or tablet.

3. The solid composition of claim 1, wherein the first enzyme is a reverse transcriptase enzyme, optionally wherein the reverse transcriptase enzyme is a heat-sensitive reverse transcriptase enzyme.

4-5. (canceled)

6. The solid composition of claim 1, wherein the second enzyme is a polymerase enzyme, optionally wherein the polymerase enzyme is a heat-stable polymerase enzyme.

7-8. (canceled)

9. The solid composition of claim 1, wherein the first enzyme and second enzyme are under the control of the same molecular switch, optionally wherein the molecular switch comprises an aptamer binding site, an antibody binding site, or a photocleavable site.

10. (canceled)

11. The solid composition of claim 1, further comprising an inactivating agent bound to the molecular switch.

12. The solid composition of claim 11, wherein the inactivating agent comprises an aptamer or an antibody.

13. The solid composition of claim 12, wherein the aptamer is a polynucleotide aptamer or a peptide aptamer.

14. The solid composition of claim 1, wherein the first enzyme and second enzyme are each under the control of a different molecular switch optionally wherein the molecular switch of the first enzyme comprises an aptamer binding site, an antibody binding site, or a photocleavable site or wherein the molecular switch of the second enzyme comprises an aptamer binding site, an antibody binding site, or a photocleavable site.

15-16. (canceled)

17. The solid composition of claim 14, further comprising an inactivating agent bound to each molecular switch.

18. The solid composition of claim 17, wherein each inactivating agent is independently selected from the group consisting of an aptamer and an antibody.

19. The solid composition of claim 18, wherein each aptamer is a polynucleotide aptamer or a peptide aptamer.

20. The solid composition of claim 1, further comprising a third enzyme, optionally wherein the third enzyme is a Uracil-DNA glycosylase (UDG) enzyme, further optionally wherein the UDG enzyme is heat-sensitive.

21-25. (canceled)

26. A composition comprising the solid composition of claim 1 and a biological sample comprising DNA or RNA.

27-33. (canceled)

34. A single-tube method for amplification of a target nucleic acid, the method comprising:
(i) contacting the solid composition of claim 1 with a biological sample comprising nucleic acids to produce a reaction mixture;
(ii) incubating the reaction mixture under conditions under which uracil-containing nucleotides are removed from the nucleic acids in the reaction mixture by a UDG enzyme;
(iii) incubating the reaction mixture under conditions under which the UDG enzyme is inactivated and the molecular switches of the first and/or second enzymes of the solid composition are activated;
(iv) amplifying a target nucleic acid from the reaction mixture using the first and/or second enzymes.

35. The method of claim 34, wherein step (i) further comprises contacting the solid composition with one or more of the following to produce the reaction mixture:
one or more buffering agents, one or more oligonucleotide primers, and a population of deoxyribonucleotide triphosphates (dNTPs).

36. The method of claim 34, wherein the biological sample
(i) comprises blood, saliva, mucus, urine, feces, cerebrospinal fluid (CSF), or tissue;
(ii) comprises DNA or RNA, optionally wherein the DNA or DNA is derived from one or more pathogens.

37-39. (canceled)

40. The method of claim 34, wherein the conditions of step (ii) comprise incubating the reaction mixture at a temperature ranging from about 30° C. to about 40° C.; or wherein the conditions of step (iii) comprise incubating the reaction mixture at a temperature ranging from about 50° C. to about 70° C.

41. (canceled)

42. The method of claim 34, wherein the UDG enzyme is
(i) a component of the solid composition:,
(ii) heat-sensitive;
(iii) a bacterial UDG or a mammalian UDG; or
(iv) an E. coli UDG.

43-45. (canceled)

46. A kit comprising:
(i) a container housing the solid composition of claim 1;
(ii) a container housing one or more buffering agents;
(iii) a container housing one or more oligonucleotide primers; and
(iv) a container housing a population of deoxyribonucleotide triphosphates (dNTPs).

47-50. (canceled)
